(19) United States
(12) Patent Application Publication
(10) Pub. No.: US 2004/0156870 A1
(43) Pub. Date: Aug. 12, 2004

(54) Use of urea as a method for improving the effectiveness of topical anti-inflammatory drugs

(75) Inventors: Bradley P. Glassman, Fairfield, NJ (US); Dileep Bhagwat, Bronxville, NY (US); Daniel Glassman, Fairfield, NJ (US)

Correspondence Address: COVINGTON & BURLING, ATTN: PATENT DOCKETING, 1201 PENNSYLVANIA AVENUE, N.W., WASHINGTON, DC, 20004-2401, US

(73) Assignee: Bradley Pharmaceuticals, Inc.

(21) Appl. No.: 10/365,249

(22) Filed: Feb. 11, 2003

(51) Int. Cl. A61K 31/573; A61K 9/00

(52) U.S. Cl. 424/400; 514/179

(57) ABSTRACT

A method of improving the effectiveness of topical anti-inflammatory drug(s) in the treatment of inflammatory conditions of the skin in humans is described which includes administration of an effective amount of urea in a topical formulation to an affected area of the skin of a patient either before, after or with the administration to such affected area of an effective amount of topical anti-inflammatory drug(s). Also described are topical compositions containing a combination of urea and an anti-inflammatory agent.

### FIELD OF THE INVENTION

The present invention relates to the use of urea as a method for improving the effectiveness of topical anti-inflammatory drugs in the treatment of inflammatory conditions of the skin in humans.

### BACKGROUND OF THE INVENTION

Urea has been long recognized as a cosmetic ingredient in formulations acting as a humectant and moisturizer. High concentrations of urea, such as 40%, are also known to have mild, antibacterial effect. At these strengths the antibacterial effects are said to be similar to those of antibiotics, with the further advantage that all the common organisms are susceptible and the possibility of resistant strains need not be seriously considered. There have been reports of keratolytic activity attributed to urea with the ability at high concentrations to solubilize and denature protein. Dermatological compositions containing from 21 to 40 wt-% urea for treating dry scaly skin have been described in U.S. Pat. No. 5,919,470.

Concentrated solutions of urea can change the conformation of protein molecules. A striking effect is upon the water-binding capacity of the horny layer of the skin: pieces of normal horny layer, or scales from ichthyotic or psoriatic skin that have been soaked in 30% urea solution take up much more water. This is important because in maintaining the flexibility of the horny layer and the softness of the skin, the water content of the horny layer matters much more than its oil content.

Systemic anti-inflammatory drug therapy is associated with potentially harmful side effects. Moreover, since oral anti-inflammatory drugs, for example corticosteroids, are distributed throughout the entire body, systemic side effects such as elevated liver enzymes, gastrointestinal disorders and skin rashes are not uncommon and may require expensive medical intervention and laboratory tests. Accordingly, topical formulations for treating inflammatory conditions of the skin in humans are increasingly recommended.

### SUMMARY OF THE INVENTION

The present invention relates to the use of urea as a method for improving the effectiveness of topical anti-inflammatory drugs in the treatment of inflammatory conditions of the skin in humans. These methods include topically administering to the affected area of human skin a safe and effective amount of urea either before, after or with a safe and effective amount of a topical anti-inflammatory drug.

Accordingly, the present invention provides a method for therapeutically improving the effectiveness and manner by which topical anti-inflammatory drugs are utilized to treat inflammatory conditions of the skin in humans by topically administering a safe and effective amount of urea in a pharmaceutically acceptable carrier either before, after or with a safe and effective amount of a topical anti-inflammatory drug.

Another embodiment of the invention is a topical anti-inflammatory composition containing a combination of an effective amount of urea and a safe and effective amount of an anti-inflammatory agent together with a pharmaceutically acceptable carrier.

### DETAILED DESCRIPTION OF THE INVENTION

The present invention provides a method for improving the effectiveness of topical anti-inflammatory drugs in the treatment or prevention of inflammatory conditions of the skin. Such method includes administering to the affected area of the skin of a human in need of such treatment or prevention a safe and effective amount of urea, for example, from about 5 to 60 wt-%, preferably about 10 to 40 wt-%, and particularly about 10 wt-%, either before, after or with the administration to the affected area of a safe and effective amount of a topical anti-inflammatory drug.

The terms “administering” or “administration”, as needed herein, refer to any method which, in sound medical practice, delivers the urea either before, after or with the delivery of a safe and effective amount of a topical anti-inflammatory drug in such a manner so as to be effective in the treatment of inflammatory conditions of the skin. Preferably, the urea and the anti-inflammatory drug(s) is administered topically in a single composition.

The phrase “safe and effective amount”, as used herein, means an amount of urea sufficient enough to significantly and positively modify the condition to be treated but low enough to avoid serious side effects, within the scope of sound medical advice. The safe and effective amount of the urea of the present invention to be utilized either before, after or with the delivery of a safe and effective amount of a topical anti-inflammatory drug(s) will vary with the particular condition being treated, the severity of the condition, the duration of treatment, the particular pharmaceutically acceptable carriers utilized, and the like factors within the knowledge and expertise of the attending physician.

The method of the present invention typically involves administering the urea either before, after or with the delivery of a safe and effective amount of a topical anti-inflammatory drug(s) in an amount to cover the affected area. The specific preferred quantity of the urea depends upon the characteristics of the anti-inflammatory it is being used in conjunction with, together with the nature of the inflammation and other skin conditions present.

For the method of the present invention, the duration of administration of the urea either before, after or with a topical anti-inflammatory drug(s) will vary according to the specific extent of the inflammatory condition being treated, but typically is within the range of 1 to 60 days. Reapplication of the urea and anti-inflammatory drug(s) may be necessary as the medical condition warrants and at the direction of the attending physician.

### Topical Anti-Inflammatory Agents

Inflammation is a local response to cellular injury that is marked by capillary dilatation, leukocytic infiltration, redness, heat, and pain and that serves as a mechanism initiating the elimination of noxious agents and of damaged tissue. A human or animal must defend itself against multitude of different pathogens including viruses, bacteria, fungi, and protozoan and metazoan parasites as well as tumors and a number of various harmful agents which are capable to derange its homeostasis. For this, a plenty of effector mechanisms capable of defending the body against such antigens and agents have developed and these can be mediated by soluble molecules or by cells. If infection occurs as a consequence of the tissue damage, the innate and, later, the adaptive immune systems are triggered to destroy the infectious agent.

Inflammation is a complex stereotypical reaction of the body expressing the response to damage of its cells and vascularized tissues. In avascular tissues, e.g. in normal cornea, the true inflammation does not occur.

The discovery of the detailed processes of inflammation has revealed a close relationship between inflammation and the immune response.

The five basic symptoms of inflammation—redness (rubor), swelling (tumor), heat (calor), pain (dolor) and deranged function (functio laesa) have been known since the ancient Greek and Roman era. These signs are due to extravasation of plasma and infiltration of leukocytes into the site of inflammation. Early investigators considered inflammation a primary host defense system. From this point of view inflammation is the key reaction of the innate immune response but in fact, inflammation is more than this, since it can lead to death, as in anaphylactic shock, or debilitating diseases, as in arthritis and gout.

According to different criteria, inflammatory responses can be divided into several categories. The criteria include:

1. time—hyperacute (peracute), acute, subacute, and chronic inflammation;

2. the main inflammatory manifestation—alteration, exudation, proliferation;

3. the degree of tissue damage—superficial, profound (bordered, not bordered);

4. characteristic picture—nonspecific, specific;

5. immunopathological mechanisms

allergic (reaginic) inflammation,

inflammation mediated by cytotoxic antibodies,

inflammation mediaded by immune complexes,

delayed-type hypersensitivity reactions.

Inflammation is the body's reaction to invasion by an infectious agent, antigen challenge or even just physical, chemical or traumatic damage.

The mechanism for triggering the response the body to injury is extremely sensitive. Responses are to tissue damage that might not normally be thought of as injury, for example when the skin is stroked quite firmly or if some pressure is applied to a tissue. In addition, the body has the capacity to respond to both minor injuries such as bruising, scratching, cuts, and abrasions, as well as to major injuries such as severe burns and amputation of limbs.

Depending on the severity of the tissue damage resulting from an injury, the integrity of the skin or internal surfaces may be breached and damage to the underlying connective tissue and muscle, as well as blood vessels can occur. In this situation infection can, and frequently does result because the normal barrier to the entry of harmful organisms has been broken. It is obviously most important that the body can respond to injury by healing and repairing the damaged tissue, as well as by eliminating the infectious agents that may have entered the wound and their toxins. It is also important that the appropriate response to the tissue damage and infection can be made: it is no use bringing all of the body's defenses into action to repair a minor scratch, just as one would not expect a single mechanism to be able to deal with the sudden loss of a limb or a major infection.

The inflammatory reaction is phylogenetically and ontogenetically the oldest defense mechanism. The cells of the immune system are widely distributed throughout the body, but if an infection or tissue damage occurs it is necessary to concentrate them and their products at the site of damage. Three major events occur during this response:

1. An increased blood supply to the tissue “in danger”. It is performed by vasodilation. The inflamed tissue looks like containing greater number of vessels.

2. Increased capillary permeability caused by retraction of the endothelial cells. This permits larger molecules than usual to escape from the capillaries, and thus allows the soluble mediators of immunity to reach the site of inflammation.

3. Leukocytes migrate out of the capillaries into the surrounding tissues. In the earliest stages of inflammation, neutrophils are particularly prevalent, but later monocytes and lymphocytes also migrate towards the site of infection.

For the possibility of surrounding tissue damage, inflammatory responses must be well ordered and controlled. The body must be able to act quickly in some situations, for example to reduce or stop the lost of blood, whereas tissue repair and reconstruction can begin a little later. Therefore, a wide variety of interconnected cellular and humoral (soluble) mechanisms are activated when tissue damage and infection occur. On the other hand if the injury is negligible, the body must have mechanisms which are able to stop the tissue damage when the injury agent was removed.

The development of inflammatory reactions is controlled by cytokines, by products of the plasma enzyme systems (complement, the coagulation clothing, kinin and fibrinolytic pathways), by lipid mediators (prostaglandins and leukotrienes) released from different cells, and by vasoactive mediators released from mast cells, basophils and platelets. These inflammatory mediators controlling different types of inflammatory reaction differ. Fast-acting mediators, such as vasoactive amines and the products of the kinin system, modulate the immediate response. Later, newly synthesized mediators such as leukotrienes are involved in the accumulation and activation of other cells. Once leukocytes have arrived at a site of inflammation, they release mediators which control the later accumulation and activation of other cells.

However, in inflammatory reactions initiated by the immune system, the ultimate control is exerted by the antigen itself, in the same way as it controls the immune response itself. For this reason, the cellular accumulation at the site of chronic infection, or in autoimmune reactions (where the antigen cannot ultimately be eradicated), is quite different from that at sites where the antigenic stimulus is rapidly cleared.

Anti-inflammatory compositions known to be useful for the treatment of inflammatory conditions of the skin include, but are not limited to: topical creams, ointments, solutions, foams, lacquers and gels containing as active anti-inflammatory agents, for example, corticosteroids and nonsteroidal anti-inflammatory agents (NSAIDs). Examples of corticosteroids include, but are not limited to, betamethasone, clobetasol, diflorasone, halobetasol, amcinonide, desoximetasone, diflorasone, fluocinolone, fluocinonide, halcinonide, triamcinolone, clocortolone, desoximetasone, flurandrenolide, fluticasone, hydrocortisone, mometasone, aclometasone, desonide, and dexamethasone. Examples of nonsteroidal anti-inflammatory drugs (NSAIDs) are flurbiprofen, diclofenac, metronidazole, ketorolac and their pharmaceutically acceptable salts.

Additional keratolytic agents such as salicylic acid and alpha hydroxy acids can be included in the composition.

We have now surprisingly found urea potentiates the effect of known anti-inflammatory agents, when used with topical anti-inflammatory agents. Moreover, we have now found that the use of urea, either before, after or with the use of a topical anti-inflammatory drugs, increases the effectiveness of topical anti-inflammatory in the treatment of inflammatory conditions of the skin. Urea was previously known for its effectiveness for tissue softening and treating dry skin, without the need of traditional preservatives. Urea was also reported to have a mild antibacterial effect. However, nothing would have suggested that urea could significantly enhance the effects of topically applied anti-inflammatory agents.

Thus, the present invention provides a method for improving the effectiveness of topical anti-inflammatory drug(s) in the treatment of inflammatory conditions of the skin by utilizing a topical composition containing urea either before, after or with the use of a topical anti-inflammatory drug(s).

When a topical anti-inflammatory agent is administered with urea, it may be administered in a single composition. This topical composition contains a combination of varied amounts of urea and anti-inflammatory agents. For example, the amount of urea may vary from 1 to 60 wt-%, or from 10 to 40 wt-%. The amount of anti-inflammatory agent compatible with the urea in a topical composition may range from about 0.01 to 10 wt-%, preferably 0.05 to 5 wt-%, for a corticosteroid, and from about 0.5 to 40 wt-% for an NSAID.

In addition to containing a therapeutically effective amount of urea and anti-inflammatory agents, the composition includes a pharmaceutical carrier, such as dermatologically acceptable excipients as described in U.S. Pat. No. 5,919,470, which patent is incorporated herein by reference.

The above excipients and active ingredients as topical compositions are manufactured by known methods into creams, ointments, solutions, lacquers, gels, foams, and any other vehicle that can be applied directly to the affected area of skin.

Typical compositions containing urea employed in the present invention are for example:

| Ingredient | Approximate Wt-% |
|---|---|
| Anti-inflammatory drug (corticosteroid) | 0.01-5 |
| urea | 10-40 |
| petrolatum or a synthetic or semi-synthetic | 5.5-20 |
| hydrocarbon, or a semi-solid mixture thereof | |
| liquid petrolatum or synthetic or semi-synthetic | 10-20 |
| oleaginous liquid fraction, or a mixture thereof | |
| C₁₆₋₁₈ aliphatic straight or branched chain fatty | 0.25-2 |
| alcohol or fatty acid, or a mixture thereof | |
| propylene glycol | 1-5 |
| glyceryl stearate | 1-3 |
| xanthan gum | 0.01-0.5 |
| water Qs | 100.00 |
| Anti-inflammatory drug (NSAID) | 0.5-40 |
| urea | 10-40 |
| petrolatum or a synthetic or semi-synthetic | 5.5-20 |
| hydrocarbon, or a semi-solid mixture thereof | |
| liquid petrolatum or a synthetic or semi-synthetic | 10-20 |
| oleaginous liquid fraction, or a mixture thereof | |
| C₁₆₋₁₈ aliphatic straight or branched chain fatty | 0.25-2 |
| alcohol or fatty acid, or a mixture thereof | |
| propylene glycol | 1-5 |
| glyceryl stearate | 1-3 |
| xanthan gum | 0.01-0.5 |
| mixture of a carbomer and triethanolamine | 0.05-30 |
| | (optional) |
| water Qs | 100.00 |

### EXAMPLES

### Example 1

The following cream was prepared using hydrocortisone and urea as active ingredients. The ingredients shown below were mixed together to form a cream according to well known pharmaceutical manufacturing methods.

| Ingredient | % W/W |
|---|---|
| Hydrocortisone | 1.020 |
| Urea USP | 10.200 |
| Carbopol 940 | 0.400 |
| Stearic acid | 10.00 |
| Propylene glycol | 3.600 |
| Isopropyl myristate | 4.000 |
| Isopropyl palmitate | 4.000 |
| PPG-26 oleate | 4.000 |
| Sodium laureth sulfate | 0.064 |
| Sodium metabisulfite | 0.300 |
| Cetyl alcohol | 0.500 |
| Edetate disodium | 0.10 |
| Xanthan gum | 0.030 |
| Trolamine | 0.750 |
| Purified Water QS | 100.000 |

### Example 2

A double-blind, bilateral, paired-comparison study was conducted on 70 patients with atopic dermatitis. Active and symmetrical dermatitis was present in all patients. Two formulations were tested. One formulation contained 1% hydrocortisone acetate alone and a second formulation contained 1% hydrocortisone acetate with 10% urea both in a cream base. The clinical data showed that the urea plus 1% hydrocortisone acetate formulation produced more than three times the effectiveness in relieving inflammation than the hydrocortisone acetate formulation without urea. When the data for patients in the 1-19 year old groups were evaluated, urea plus 1% hydrocortisone acetate was about six times more effective in relieving inflammation when compared to the hydrocortisone acetate formulation without urea.

We claim:

1. A method for improving the effectiveness of topical anti-inflammatory drug(s) in the treatment of inflammatory conditions of the skin comprising administering to the affected area of a patient in need thereof a composition comprising a therapeutically effective amount of urea and the balance being dermatologically acceptable excipients either before, after or with the administration to the affected area of such patient a therapeutically effective amount of a topical anti-inflammatory drug.

2. The method of claim 1, wherein the anti-inflammatory drug(s) is a corticosteroid.

3. The method of claim 2, wherein the effective amount of corticosteroid is from about 0.01 to about 10 wt-%.

4. The method of claim 1, wherein the anti-inflammatory drug(s) is a non-steroidal anti-inflammatory drug.

5. The method of claim 4, wherein the effective amount of the non-steroidal anti-inflammatory drug is from about 0.5 to about 40 wt-%.

6. The method of claim 1, wherein the composition comprises from about 5 to about 60 wt-% urea.

7. The method of claim 1, wherein the composition comprises from about 10 to 40 wt-% urea.

8. The method of claim 1, wherein the composition is selected from the group consisting of topical creams, ointments, solutions, lacquers, gels, foams and any other vehicle that can be applied directly to the affected area of the skin.

9. A method for improving the effectiveness of topical anti-inflammatory drug(s) in the treatment of inflammatory conditions of the skin comprising administering to the affected area of a patient in need thereof a composition comprising about 10 to about 40 wt-% urea and about 0.01 to about 5% of a corticosteroid or about 0.5 to about 40 wt-% of a non-steroidal anti-inflammatory drug formulated in a pharmaceutically acceptable topical base.

10. A topical anti-inflammatory composition comprising:
(a) a safe and effective amount of urea;
(b) a safe and effective amount of an anti-inflammatory agent; and
(c) a balance of dermatologically acceptable excipients.

11. The composition of claim 10 comprising:
(a) from about 10 to about 40 wt-% urea;
(b) from about 0.01 to about 5 wt-% of a corticosteroid; and
(c) the balance being dermatologically acceptable excipients.

12. The composition of claim 10 comprising:
(a) from about 10 to about 40 wt-% urea;
(b) from about 0.5 to about 40 wt-% of a non-steroidal anti-inflammatory agent; and
(c) the balance being dermatologically acceptable excipients.

13. The composition of claim 10 comprising:
(a) from about 10 to about 40 wt-% urea;
(b) from about 0.01 to about 5 wt-% of a corticosteroidal anti-inflammatory agent; and
(c) from about 5.5 to about 20 wt-% petrolatum or a synthetic or semi-synthetic hydrocarbon, or a semi-solid mixture thereof;
(d) from about 10 to about 20 wt-% liquid petrolatum or synthetic or semi-synthetic oleaginous liquid fraction, or a mixture thereof;
(e) from about 0.25 to 2 wt-% of a C₁₆₋₁₈ aliphatic straight or branched chain fatty alcohol or fatty acid, or a mixture thereof,
(f) from about 1 to about 5 wt-% propylene glycol;
(g) from about 1 to about 3 wt-% glyceryl stearate;
(h) from about 0.01 to about 0.5 wt-% xanthan gum; and
(i) the balance being water.

14. The composition of claim 10 comprising:
(a) from about 10 to about 40 wt-% urea;
(b) from about 0.5 to about 40 wt-% of a non-steroidal anti-inflammatory agent;
(c) from about 5.5 to about 20 wt-% petrolatum or a synthetic or semi-synthetic hydrocarbon, or a semi-solid mixture thereof;
(d) from about 10 to about 20 wt-% liquid petrolatum or synthetic or semi-synthetic oleaginous liquid fraction, or a mixture thereof,
(e) from about 0.25 to 2 wt-% of a C₁₆₋₁₈ aliphatic straight or branched chain fatty alcohol or fatty acid, or a mixture thereof;
(f) from about 1 to about 5 wt-% propylene glycol;
(g) from about 1 to about 3 wt-% glyceryl stearate;
(h) from about 0.01 to about 0.5 wt-% xanthan gum; and
(i) the balance being water.

15. The composition of claim 10 comprising:
(a) from about 10 to about 40 wt-% urea;
(b) from about 0.01 to about 5 wt-% of a corticosteroidal anti-inflammatory agent;
(c) from about 5.5 to about 20 wt-% petrolatum or a synthetic or semi-synthetic hydrocarbon, or a semi-solid mixture thereof;
(d) from about 10 to about 20 wt-% liquid petrolatum or synthetic or semi-synthetic oleaginous liquid fraction, or a mixture thereof;
(e) from about 0.25 to 2 wt-% of a C₁₆₋₁₈ aliphatic straight or branched chain fatty alcohol or fatty acid, or a mixture thereof;
(f) from about 1 to about 5 wt-% propylene glycol;
(g) from about 1 to about 3 wt-% glyceryl stearate;
(h) from about 0.01 to about 0.5 wt-% xanthan gum;
(i) from about 0.05 to about 30 wt-% of a mixture of a carbomer and triethanolamine; and
(j) the balance being water.

16. The composition of claim 10, comprising:
(a) from about 10 to about 40 wt-% urea;
(b) from about 0.5 to about 40 wt-% of a non-steroidal anti-inflammatory agent;
(c) from about 5.5 to about 20 wt-% petrolatum or a synthetic or semi-synthetic hydrocarbon, or a semi-solid mixture thereof;
(d) from about 10 to about 20 wt-% liquid petrolatum or synthetic or semi-synthetic oleaginous liquid fraction, or a mixture thereof;
(e) from about 0.25 to 2 wt-% of a C₁₆₋₁₈ aliphatic straight or branched chain fatty alcohol or fatty acid, or a mixture thereof;
(f) from about 1 to about 5 wt-% propylene glycol;
(g) from about 1 to about 3 wt-% glyceryl stearate;
(h) from about 0.01 to about 0.5 wt-% xanthan gum;
(i) from about 0.05 to about 30 wt-% of a mixture of a carbomer and triethanolamine; and
(j) the balance being water.
